(19) United States
(12) Patent Application Publication
(10) Pub. No.: US 2024/0084142 A1
(43) Pub. Date: Mar. 14, 2024

(54) SUBSTRATE FOR DIFFUSING VOLATILE SUBSTANCES

(71) Applicant: Zobele Holding SPA, Trento (IT)

(72) Inventors: Sergio LUQUE VERA, Barcelona (ES); Alberto José SANCHO MARZO, Barcelona (ES); María GARCÍA SUBIRATS, Barcelona (ES); David ALONSO CAÑADAS, Barcelona (ES)

(73) Assignee: Zobele Holding SPA, Trento (IT)

(21) Appl. No.: 18/270,003

(22) PCT Filed: Dec. 23, 2021
(86) PCT No.: PCT/EP2021/087459

§ 371 Date: Jun. 28, 2023

(30) Foreign Application Priority Data
Dec. 28, 2020 (ES) P202031303

(51) Int. Cl. C08L 97/02; C08K 5/098

(52) U.S. Cl. CPC C08L 97/02; C08K 5/098; C08L 2201/06; C08L 2201/08

(57) ABSTRACT

The substrate for diffusing volatile substances comprises a dispersible binder and a biodegradable material, the percentages by weight on the dry state being preferably: dispersible binder: 15-45% and biodegradable material: 55-75%. It may also comprise a biodegradable preservative such as sodium benzoate. It permits to provide a biodegradable substrate to diffuse volatile substances, which has a short biodegradation time.

The present invention relates to a substrate for diffusing volatile substances, which has a short biodegradation time.

### BACKGROUND OF THE INVENTION

Sustainability is clearly a particularly important criterion for consumers today when deciding which products to buy.

Among the different proposals to improve sustainability, biodegradability is a message that consumers perceive particularly well. It is believed that a biodegradable product discarded in nature will spontaneously disintegrate and leave no trace. Unfortunately, this is a misunderstanding of what biodegradable means.

Biodegradable materials are such that they can be disintegrated by bacteria when placed in a composting environment under fairly controlled conditions of humidity and temperature. Biodegradability may still occur to some extent under uncontrolled conditions, but understanding the controlling factors is key to fully asserting that a material is biodegradable.

Volatile substance containers are generally poorly biodegradable. The most common multi-material container applications consist of wick bottles that allow a high-volume content (15-30 ml or more) or cellulose-based tablets that allow a low initial volume content (generally less than 1 g).

There are also other porous systems thicker than cellulosic pellets, such as porous ceramics or porous plastics where the thickness of the material can reach centimeters and the volumetric capacity is much higher.

While complex multi-material containers are a sustainability challenge, cellulosic pellets themselves can be easily recycled or biodegraded. However, as discussed, they cannot contain large quantities and are generally limited to short-live insecticides and air fresheners.

Thicker materials such as porous plastics or ceramics are a problem for biodegradability, as the biodegradation time increases exponentially with thickness.

The most common applications that meet composting standards are limited to thin materials (0.01-0.5 mm), such as used packaging applications. In air diffusion applications, a substrate material used to absorb and diffuse liquids requires thicker materials (1-10 mm), making it difficult to meet composting requirements.

### DESCRIPTION OF THE INVENTION

Therefore, there is a need for a thick, porous substrate that can contain a large amount of volatile substance and still have a short biodegradation time. Ideally, not only the substrate needs to be biodegradable, but also the volatile substance, or at least the residual part of the volatile substance.

Therefore, one purpose of the present invention is to provide a biodegradable substrate for diffusing volatile substances, having a short biodegradation time.

With the substrate according to the present invention it is possible to solve said drawbacks, providing other advantages which are described below.

The substrate for diffusing volatile substances according to the present invention comprises a mixture of different biodegradable ingredients with different granularities and a dispersible binder. The substrate provides a wide range of dimensional and shape possibilities and high compostability characteristics.

The substrate is made of biodegradable particles and a dispersible binder material, such as flour-like materials, mixed with different granular grade materials, such as wood, e.g. sawdust. The composition is processed with water by conventional processes such as, for example, compression molding or extrusion. After processing, the substrate is dried to a solid state.

The substrate material in contact with the water present in the composting environments triggers its structural decomposition, eventually breaking the piece into smaller and weaker pieces, which can be easily assimilated by the compost.

As a result, the contact area between the high-quality granular material and the environment increases, providing more favorable conditions for composting.

The substrate for diffusing volatile substances according to the present invention, with the aid of a heating element or an air flow, can be labelled as a compostable material.

According to claim 1, the substrate for diffusing volatile substances according to the present invention comprises:
a dispersible binder; and
a biodegradable material.

In addition, it may also comprise a biodegradable preservative.

Advantageously, the percentages by weight in the dry state are as follows:
dispersible binder: 15-45%;
biodegradable material: 55-75%; and
biodegradable preservative: 0.1-5%.

More preferably, the percentages by weight in the dry state are as follows:
dispersible binder: 20-30%;
biodegradable material: 65-75%; and
biodegradable preservative: 0.5-1.5%.

According to a preferred embodiment, the granulometry of the dispersible binder is between 100 and 250 μm, such as 180 μm, and the granulometry of the biodegradable material is between 0 and 250 μm, such as less than 60 μm.

Preferably, the dispersible binder is flour and the biodegradable material is wood, such as wood sawdust.

In addition, the biodegradable preservative is preferably sodium benzoate.

### DESCRIPTION OF A PREFERRED EMBODIMENT

By way of example, the substrate according to the present invention has the following composition.

Granulometry (μm)

| Substrate composition | Interval | Preferential |
|---|---|---|
| Dispersible binder (flour) | 100-250 | 180 |
| Biodegradable material (wood) | 0-250 | <60 |
| Biodegradable preservative | — | — |
| Water | — | — |

| | % by weight, hydrated | | % by weight, dry | |
|---|---|---|---|---|
| Substrate composition | Interval | Preferential | Interval | Preferential |
| Dispersible binder (flour) | 5-15% | 7-10% | 15-45% | 20-30% |
| Biodegradable material | 15-30% | 20-25% | 55-75% | 65-75% |
| (wood) | | | | |
| Biodegradable | 0.1-5% | 0.5-1% | 0.1-5% | 0.5-1.5% |
| preservative | | | | |
| Water | 40-80% | 50-70% | — | — |

| | Granulometry | | % by weight, | | | |
|---|---|---|---|---|---|---|
| Substrate | range (μm) | | hydrated | | % by weight, dry | |
| composition | Interval | Preferential | Interval | Preferential | Interval | Preferential |
| Dispersible | 100-250 | 180 | 5-15% | 7-10% | 15-45% | 20-30% |
| binder (flour) | | | | | | |
| Biodegradable | 0-250 | <60 | 15-30% | 20-25% | 55-75% | 65-75% |
| material (wood) | | | | | | |
| Biodegradable | — | — | 0.1-5% | 0.5-1% | 0.1-5% | 0.5-1.5% |
| preservative | | | | | | |
| Water | — | — | 40-80% | 50-70% | — | — |

Following the requirements of EN 13432, 90% of the substrate must degrade within 6 months. Therefore, the formulation must be composed of a minimum of 90% biodegradable ingredients.

Furthermore, according to a preferred embodiment, the substrate according to the present invention is disc-shaped, although it may have a different shape, and its thickness varies from 0.1 to 20 mm.

A non-limiting example of substrate composition is:
Dispersible binder (flour): 25.8% by weight on dry state
Biodegradable material (wood): 73.2% by weight on dry state
Biodegradable preservative (sodium benzoate): 1% by weight on dry state

Although reference has been made to a specific embodiment of the invention, it is obvious to a person skilled in the art that the substrate described herein is susceptible to numerous variations and modifications, and that all the details mentioned can be replaced by technically equivalent ones without departing from the scope of protection defined by the appended claims.

1. A substrate for diffusing volatile substances, characterized in that the substrate comprises:
a dispersible binder; and
a biodegradable material.

2. The substrate for diffusing volatile substances according to claim 1, wherein the percentages by weight on the dry state are as follows:
dispersible binder: 15-45%; and
biodegradable material: 55-75%.

3. The substrate for diffusing volatile substances according to claim 2, wherein the percentages by weight on the dry state are as follows:
dispersible binder: 20-30%; and
biodegradable material: 65-75%.

4. The substrate for diffusing volatile substances according to claim 1, also comprising a biodegradable preservative.

5. The substrate for diffusing volatile substances according to claim 4, wherein the percentage by weight on dry state of the biodegradable preservative is between 0.1 and 5%, in particular between 0.5 and 1.5%.

6. The substrate for diffusing volatile substances according to claim 1, wherein the granulometry of the dispersible binder is between 100 and 250 μm.

7. The substrate for diffusing volatile substances according to claim 6, wherein the granulometry of the dispersible binder is 180 μm.

8. The substrate for diffusing volatile substances according to claim 1, wherein the granulometry of the biodegradable material is between 0 and 250 μm.

9. The substrate for diffusing volatile substances according to claim 8, wherein the granulometry of the biodegradable material is less than 60 μm.

10. The substrate for diffusing volatile substances according to claim 1, wherein the dispersible binder is flour.

11. The substrate for diffusing volatile substances according to claim 1, wherein the biodegradable material is wood.

12. The substrate for diffusing volatile substances according to claim 11, wherein the biodegradable material is wood sawdust.

13. The substrate for diffusing volatile substances according to claim 4, wherein the biodegradable preservative is sodium benzoate.
